(19) United States
(12) Patent Application Publication
(10) Pub. No.: US 2006/0154861 A1
(43) Pub. Date: Jul. 13, 2006

(54) Method for screening substances capable of inhibiting the hyperplasia of pancreatic cells and/or the hypersecretion of insulin by pancreatic cells

(75) Inventors: Akemichi Baba, Hyogo (JP); Hitoshi Hashimoto, Osaka (JP); Norihito Shintani, Osaka (JP); Yukihiko Sugimoto, Kyoto (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Taisho Pharmaceutical Co., Ltd., Toshima-ku (JP); Akemichi BABA, Nishinomiya-shi (JP)

(21) Appl. No.: 11/257,107

(22) Filed: Oct. 25, 2005

(30) Foreign Application Priority Data
Jan. 12, 2005 (JP) JP2005-004893

(51) Int. Cl. A61K 38/22

(52) U.S. Cl. 514/12

(57) ABSTRACT

There are provided PACAP which is a substance capable of inhibiting the hyperplasia of pancreatic cells and/or the hypersecretion of insulin by pancreatic cells, a method for inhibiting the hyperplasia of pancreatic cells and/or the hypersecretion of insulin by pancreatic cells in a human by PACAP administration, a pharmaceutical composition containing PACAP and, optionally, an anti-diabetic agent, and a method for evaluating a substance capable of inhibiting the hyperplasia of pancreatic cells and/or the hypersecretion of insulin by pancreatic cells relying on the expression of Reg IIIβ to be stimulated by PACAP as an index.

### BACKGROUND OF THE INVENTION

1. Field of the Invention

This invention relates to an inhibitor of the hyperplasia of pancreatic cells and/or an inhibitor of the hypersecretion of insulin by pancreatic cells, to a method for screening a substance capable of inhibiting the hyperplasia of pancreatic cells and/or the hypersecretion of insulin by pancreatic cells and to a substance capable of inhibiting the hypersecretion of insulin by pancreatic cells obtained by the screening method.

2. Related Background Art

Pituitary adenylate cyclase-activating polypeptide (PACAP) is a neuropeptide belonging to the secretin/glucagon/vasoactive intestinal polypeptide (VIP) family. It has been known that the physiological function of the peptide is responsible for diverse roles such as the regulating actions on hormonal synthesis and secretion in pituitary and adrenal medulla, and the differentiation and growth-promoting actions of nerve cells and germ cells (“Bessatsu, Igakunoayumi-Advances in Medicine, Supplement Edition,” 2001, pp. 79-84”). It has been known that: (a) PACAP immuno-positive nerve projects into islets; (b) the expressions of a PAC₁ receptor displaying high affinity to PACAP among PACAP receptor subtypes and a VPAC₂ receptor displaying nearly equal affinities to both of PACAP and VIP are observed in pancreatic β cells; and (c) PACAP promotes the glucose-inducible insulin secretion by the isolated islet at an extremely low level of 10⁻¹⁴ M.

Since PACAP possesses cytoprotection activity or the promoting activity of cell differentiation and growth in various types of cells, it is predicted that PACAP will display such activities in the pancreatic β cells as well. However, as stated above, PACAP has diverse activities in vivo; therefore, in the experiments where sugar metabolism was examined upon the PACAP administration, the interpretation of the obtained results proved to be complicated (Am J Physiol 1998 May 274 (5 Pt1): E834-42).

Recently, in order to study the type of the activity that PACAP possesses in the pancreas, a transgenic mouse (Tg/+) was constructed where PACAP was caused to overexpress in a pancreas-specific manner under the control of the human insulin promoter. It was then discovered that this mouse displayed insulin-secreting ability in a glucose-dependent manner which was higher than that with a wild-type mouse (+/+) from the brood (Diabetes (2003) 52: 1155-1162).

Further, an agouti yellow mouse (Ay/+) that would develop obesity and diabetes in a genetically dominant manner was crossed with a Tg/+ mouse to produce a Tg/+:Ay/+ mouse. It was then discovered that the Tg/+:Ay/+ mouse experienced an increase in the pancreas weight with a decrease in the islet weight (JP2003304778A).

However, since any selective low molecular weight agonist has not been identified to exist in the PACAP signal system, there is much that has not been uncovered in respect of the PACAP action on the islets in vivo.

Regenerating genes (Reg) were isolated as genes that were specifically expressed in regenerating islets and were considered the regenerating-growth factor of β cells [Diabetes (1984) 33: 401]. It was reported that there were four subtypes (1-IV) of Reg and that Reg IIIβ was found as a factor expressing in the regenerated islets and was involved in the regeneration of islets [J Biol Chem (1988) 263: 2111-2114]. Recently, Reg IIIβ has also been reported to be a factor induced mainly by stress and inflammation [World J Gastroenterol (2003) 9: 2635-2641].

It was reported that Reg receptors were expressed in normal and regenerated islets and other sites, and that the signals of regeneration and growth were mediated by the Reg receptors [(J Biol Chem (2000) 275: 10723-10726)].

However, there has been no report on any factor capable of inhibiting the hyperplasia of pancreatic cells that is responsible for the hypofunction or dedifferentiation of islets.

### SUMMARY OF THE INVENTION

This invention resides in providing a substance capable of inhibiting the hyperplasia of pancreatic cells that is responsible for the hypofunction or dedifferentiation of islets or a substance capable inhibiting the hypersecretion of insulin by pancreatic cells. Another aspect of this invention resides in providing a method for conveniently evaluating a substance capable of inhibiting the hyperplasia of pancreatic cells and/or the hypersecretion of insulin by pancreatic cells with accuracy as well as in providing an evaluation kit for performing the evaluation.

As a result of intensive and diligent studies to solve the aforementioned problems, the present inventors discovered that PACAP induced the expression of the Reg genes in the presence of high concentration glucose and inhibited the hyperplasia of pancreatic cells and/or the hypersecretion of insulin by pancreatic cells. Moreover, the present inventors discovered that the Reg genes which had been implicated in the participation of the regeneration/growth of islets were unexpectedly involved in the inhibition of hyperplasia of pancreatic cells under the conditions of high concentration glucose and that based on this finding the expression of a Reg gene under the conditions of high concentration glucose could be made an index when evaluation would be carried out on any substance capable of inhibiting the hyperplasia of pancreatic cells and/or the hypersecretion of insulin by pancreatic cells. This invention has been accomplished upon these findings.

Specifically, this invention provides:

(1) A method for inhibiting the hyperplasia of pancreatic cells and/or the hypersecretion of insulin by pancreatic cells in a human showing such symptom and in need of inhibition thereof, the method comprises administering to the human an inhibition effective amount of pituitary adenylate cyclase-activating peptide.

(2) An inhibitor of the hyperplasia of pancreatic cells and/or the hypersecretion of insulin by pancreatic cells comprising pituitary adenylate cyclase-activating peptide.

(3) A pharmaceutical composition for inhibiting the hyperplasia of pancreatic cells and/or the hypersecretion of insulin by pancreatic cells comprising an effective amount of pituitary adenylate cyclase-activating peptide and a pharmaceutically acceptable carrier.

(4) A method for preventing or treating a patient afflicted with diabetes and in need of such treatment or a patient suspected of developing diabetes, the method comprising administering to the patient, pituitary adenylate cyclase-activating peptide and one or more agents selected from a biguanide drug, an α-glucosidase inhibitor, a thiazoline derivative or a sulfonylurea drug.

(5) The method according to (4) as described above, wherein the pituitary adenylate cyclase-activating peptide and the agent are administered simultaneously, separately or consecutively.

(6) The method according to (4) or (5) as described above, wherein the diabetes is type 2 diabetes.

(7) A prophylactic or therapeutic agent for diabetes comprising pituitary adenylate cyclase-activating peptide and one or more agents selected from a biguanide drug, an α-glucosidase inhibitor, a thiazoline derivative or a sulfonylurea drug.

(8) A pharmaceutical composition comprising pituitary adenylate cyclase-activating peptide characterized by being used simultaneously, separately or consecutively together with a pharmaceutical composition comprising as the effective agent, one or more agents selected from a biguanide drug, an α-glucosidase inhibitor, a thiazoline derivative or a sulfonylurea drug in the prevention or treatment of diabetes.

(9) A method for evaluating a substance capable of inhibiting the hyperplasia of pancreatic cells and/or the hypersecretion of insulin by pancreatic cells, the method comprising contacting a substance to be assayed with a cell expressing Reg IIIβ and employing as an index, any change in the expression of Reg IIIβ upon the contact.

(10) The method according to (9) as described above comprising the steps of:

(a) contacting the substance to be assayed with the cell expressing Reg IIIβ;

(b) reculturing the cell contacted with the substance to be assayed in step (a) in the presence of high concentration glucose; and

(c) examining the expression level of Reg IIIβ or the growth rate of the cell expressing Reg IIIβ after culturing in step (b)

(11) The method for evaluation according to (10) as described above, wherein in step (b) the culturing is carried out in the presence of pituitary adenylate cyclase-activating peptide when the substance to be assayed and the cell expressing Reg IIIβ are recultured.

(12) A kit for performing any of the methods for evaluation according to any of (9) to (11) as described above comprising a cell expressing Reg IIIβ, a reagent adapted to contacting a substance to be assayed with the cell, a reagent adapted to culturing the cell in the presence of high concentration glucose and pituitary adenylate cyclase-activating peptide.

(13) An inhibitor of the hyperplasia of pancreatic cells and/or the hypersecretion of insulin by pancreatic cells comprising a substance obtained by the method for evaluation according to any of (9) to (11) as described above.

(14) A prophylactic or therapeutic agent for diabetes comprising a substance capable of inhibiting the hyperplasia of pancreatic cells and/or the hypersecretion of insulin by pancreatic cells obtained by the method for evaluation according to any of (9) to (11) as described above and one or more agents selected from a biguanide drug, an α-glucosidase inhibitor, a thiazoline derivative or a sulfonylurea drug.

(15) The inhibitor of the hyperplasia of pancreatic cells and/or the hypersecretion of insulin by pancreatic cells according to (13) as described above characterized by being used simultaneously, separately or consecutively together with a pharmaceutical composition comprising as the effective agent, one or more agents selected from a biguanide drug, an α-glucosidase inhibitor, a thiazoline derivative or a sulfonylurea drug in the prevention or treatment of diabetes.

The pituitary adenylate cyclase-activating peptide according to this invention is useful as an inhibitor of the hyperplasia of pancreatic cells and/or the hypersecretion of insulin by pancreatic cells. According to the method for evaluation of this invention, it is possible to evaluate a substance capable of inhibiting the hyperplasia of pancreatic cells and/or the hypersecretion of insulin by pancreatic cells with ease and accuracy. This allows screening an inhibitor of the hyperplasia of pancreatic cells and/or the hypersecretion of insulin by pancreatic cells. The substance obtained by the screening can be used as the inhibitor of the hyperplasia of pancreatic cells and/or the hypersecretion of insulin by pancreatic cells. In addition, since sulfonyl drugs which are known anti-diabetic agents cause the side effect of insulin secretion disorder due to the hypofunction of pancreatic cells, they may be used as an excellent prophylactic/therapeutic agent for diabetes when they are concomitantly used with the inhibitor of the hyperplasia of pancreatic cells and/or the hypersecretion of insulin by pancreatic cells according to this invention.

### BRIEF DESCRIPTION OF THE DRAWINGS

FIGS. 1A, 1B, 1C and 1D: FIGS. 1A to 1D show the results obtained when any time-dependent changes in the body weight and the plasma components were examined in F1 mice produced by crossing a PACAP-Tg mouse (which is referred to as “Tg mouse”) with a KKA^{y} mouse (Example 2). FIG. 1A represents the body weight change; FIG. 1B represents the plasma insulin level; FIG. 1C represents the plasma glucose level; and FIG. 1D represents the plasma triglyceride level.

FIG. 2: FIG. 2 show the results obtained when hematoxylin-eosin (HE) staining was conducted on the pancreases of the F1 mice mentioned above (Example 3). The arrow in the figure shows enlargement of an islet.

FIGS. 3A, 3B, 3C and 3D: FIGS. 3A to 3D show the results obtained when the results of the hematoxylin-eosin staining conducted on the F1 mice mentioned above were further quantitatively analyzed and measured for the tissue forms (Example 3). FIG. 3A represents the mean islet area; FIG. 3B represents the number of islets per unit area of the whole pancreas; FIG. 3C represents islet mass (a value obtained by multiplying the ratio of all islets per the whole pancreas area with the wet weight of the pancreas); and FIG. 3D represents a histogram analysis of the pancreas size.

FIGS. 4A, 4B, 4C and 4D: FIGS. 4A to 4D show the results obtained when the changes in the expression of different genes in the islets of the F1 mice mentioned above were quantitatively confirmed by real-time quantitative RT-PCR (Example 5). FIG. 4A represents the expression of PACAP; FIG. 4B represents the expression of islet amyloid polypeptide (IAPP); FIG. 4C represents the expression of carboxypeptidase H; and FIG. 4D represents the expression of Reg IIIβ.

FIGS. 5A and 5B: FIGS. 5A and 5B show the results obtained when the effect of PACAP on cell growth in INS-1 cell line cells derived from pancreatic β cells was determined by a liquid scintillation counter (Example 6). FIG. 5A represents the count of cells plotted against the glucose concentration in the presence of PACAP under the conditions of different glucose concentrations. In the figure “**” indicates p<0.01. FIG. 5B represents the count of cells plotted against the logarithmic concentration at a varying PACAP concentration under the conditions of high glucose concentration. In the figure “**” indicates p<0.01.

FIGS. 6A, 6B and 6C: FIGS. 6A to 6C show the results obtained when the changes in the mRNA expression of Reg III β by PACAP in INS-1 cell line cells derived from pancreatic β cells were quantified by real-time quantitative RT-PCR (Example 6). FIG. 6A represents the time-dependent change in expression at a glucose concentration of 3 mM. FIG. 6B represents the time-dependent change in expression at a glucose concentration of 11 mM. FIG. 6C represents the time-dependent change in expression at a glucose concentration of 25 mM.

### DESCRIPTION OF THE PREFERRED EMBODIMENTS

As used in the present specification, the term “pituitary adenylate cyclase-activating polypeptide” (which will be referred to as “PACAP” hereafter) means PACAP derived from a mammal. When it is used to treat a human, it is preferably PACAP derived from a human. PACAP is a known protein and its amino acid sequence is set forth in SEQ NO:1 in the Sequence Listing.

PACAP of this invention encompasses peptides described below:

(a) A peptide comprising an amino acid sequence where one or more amino acids have been mutated in the amino acid sequence set forth in SEQ ID NO:1 by substitution, deletion, insertion and/or addition, the peptide having the same function as does the peptide represented by SEQ ID NO:1;

(b) A peptide comprising an amino acid sequence having a homology of at least 80%, preferably not less than 90%, or more preferably a sequence identity of not less than 95% with the peptide represented by SEQ ID NO:1, the peptide having the same function as does the peptide represented by SEQ ID NO:1; and

(c) A peptide encoded by a DNA sequence comprising a sequence capable of hybridizing to the anitisense segment of the DNA sequence encoding SEQ ID NO:1 under stringent conditions, the peptide having the same function as does the peptide represented by SEQ ID NO:1.

Here, the number of amino acid mutations in the peptide is preferably one or a few although it is not so limited insofar as the function of the peptide represented by SEQ ID NO:1 can be retained.

The “under stringent conditions” may appropriately be prepared according to the description of Molecular Cloning: A Laboratory Manual 3rd Ed. (2001). Specifically, such conditions include those where washing is to be done at 65° C. in a solution containing 0.1×SSC and 0.1% SDS.

As used in the present specification, the term “peptide having the same function” means a peptide having the function of enhancing the expression of Reg III after the peptide to be assayed is administered to an Reg III expressing cell cultured under the conditions of high glucose concentration and is cultured.

As used in the present specification, these peptides may be those derivable from a human or other mammals (such as a rat, mouse, rabbit, chicken, sheep, cow, and monkey) or those that were synthesized. Further, those peptides may be amide-derivatized or esterified ones.

The salts of the peptide include salts with physiologically acceptable inorganic acids (e.g., hydrochloric acid and phosphoric acid), organic acids (e.g., acetic acid, formic acid, propionic acid, fumaric acid maleic acid, succinic acid, tartaric acid, citric acid, malic acid, oxalic acid, benzoic acid, methanesulfonic acid and benzenesulfonic acid), or bases (e.g., alkaline metal). Particularly preferred are physiologically acceptable acid-addition salts.

When the peptide is administered to a human, it may be formulated into standard pharmaceutical preparations. Specifically, the peptide, a mutant thereof, a derivative thereof, or a salt thereof is compounded with pharmaceutically acceptable carriers (e.g., an excipient, binder, disintegrating agent, flavoring agent, emulsifier, diluent, and solublizer) to prepare a pharmaceutical composition. This pharmaceutical composition may be formulated into tablets, capsules, elixir or microcapsules, or alternatively, into sterilized solutions or suspensions according to conventional techniques.

As used in the present specification, the term “effective amount” means an amount or an amount of composition sufficient to exert a desired pharmacological effect (the effect to inhibit the hyperplasia of pancreatic cells and/or the hypersecretion of insulin by pancreatic cells) or a desired therapeutic or preventive effect (against diabetes) when the peptide or a salt thereof, preferably as a pharmaceutical composition, is administered to a human (a patient). When the peptide or a salt thereof is to be used for the treatment purpose, the desired pharmacological effect means that the cure or alleviation of the symptom can be achieved in the patient showing the developed symptom. When the peptide or a salt thereof is to be used for the prevention purpose, the desired pharmacological effect means the inhibition of the crisis in the patient suspected of potential crisis.

The dosage of the peptide will vary depending on the subject to be administered, the method of administration and others. For example, in the case of oral administration to a diabetic patient weighing 60 kg, the dosage will be from about 0.1 to about 100 mg per day, preferably from about 1.0 to about 50 mg, more preferably from about 1.0 to about 20 mg. In the case of parenteral administration to a diabetic patient weighing 60 kg, intravenous injection may be done at a dosage of from about 0.01 to about 30 mg per day, preferably from about 0.1 to about 20 mg, more preferably from about 0.1 to about 10 mg. The patients include those suspected of developing diabetes. The patients suspected of developing diabetes can readily be identified by the internal physicians and they are those having high blood sugar levels. (or high blood insulin levels), for example.

The peptides of this invention are endogeneous ligands existing in the body or peptides having the same function; therefore, they can be used as a safe, low toxicity inhibitor of the hyperplasia of pancreatic cells and/or the hypersecretion of insulin by pancreatic cells.

Since the peptides of this invention posses the inhibitory activity for the hyperplasia of pancreatic cells and/or the hypersecretion of insulin by pancreatic cells, they can alleviates the hypofunction of pancreatic cells resulting from the progression of diabetes.

Therefore, if the peptide of this invention is used concomitantly with a known anti-diabetic agent, it will be possible to prevent or treat diabetes while inhibiting the hyposecretion of insulin. The targeted diabetes by such treatment is, particularly, preferably type 2 diabetes.

The known anti-diabetic agents which may be used in this invention include, for example, biguanide drugs such as metformin, buformin and phenformin; α-glucosidase inhibitors such as acarbose and voglibose; thiazoline derivatives such as triglitazone and pioglitazone; and sulfonyl urea drugs such as tolbutamide, gliclazide and glibenclamide.

Particularly when the peptide is used concomitantly with an agent that acts on the pancreatic β cells and has the function of promoting the secretion of insulin (e.g., a sulfonyl urea drug), such use can reduce the hypofunction of the pancreatic cells that is generally known as the side effect of the sulfonyl urea drug and thus can be used as an excellent agent for the prevention or treatment of diabetes.

When the peptide of this invention and a known anti-diabetic agent are concomitantly used, they may be included into one preparation and administered, or alternatively, individual agents may be administered simultaneously, separately or consecutively.

The method for evaluating a substance capable of inhibiting the hyperplasia of pancreatic cells and/or the hypersecretion of insulin by pancreatic cells according to this invention may adequately be a method comprising contacting a substance to be assayed with a cell expressing Reg IIIβ and employing as an index, any change in the expression level of Reg IIIβ or in the growth rate of the cell expressing Reg III β upon the contact. When the cells expressing Reg III β are those cultured in the presence of high concentration glucose, it is possible to evaluate a substance capable of inhibiting the hyperplasia of pancreatic cells and/or the hypersecretion of insulin by pancreatic cells with higher accuracy. Specifically, the evaluation can be carried out according to the following steps:

(a) contacting the substance to be assayed with the cell expressing Reg IIIβ;

(b) reculturing the cell contacted with the substance to be assayed in step (a) in the presence of high concentration glucose; and

(c) examining the expression level of Reg IIIβ or the growth rate of the cell expressing Reg IIIβ after reculturing in step (b).

If the inhibition of the expression level of Reg IIIβ or the growth rate of the cell expressing Reg IIIβ is confirmed in step (c), the substance assayed can be considered a candidate as the inhibitor of the hyperplasia of pancreatic cells and/or the hypersecretion of insulin by pancreatic cells.

If in step (b) the reculturing is carried out in the presence of pituitary adenylate cyclase-activating peptide when the substance to be assayed and the cell expressing Reg IIIβ are recultured, the evaluation will become possible where the in vivo state is more closely reflected.

Specifically, the findings obtained in this invention can be utilized to evaluate and screen a substance capable of inhibiting the hyperplasia of pancreatic cells and/or the hypersecretion of insulin by pancreatic cells.

In this invention there can be used as the “cell expressing Reg IIIβ,” INS-1 cells (rat pancreatic βcell line), MIN-6 cells (mouse pancreatic βcell line) and the transfected cells into which DNA encoding Reg IIIβ has been inserted. The insertion of DNA encoding Reg IIIβ into the cells to be transfected may be done in cells expressing endogenous Reg IIIβ or in cells expressing no Reg IIIβ.

The insertion of the DNA encoding Reg IIIβ may be carried out by ligating the DNA to a suitable vector and introducing it to suitable cells such as mammalian cells or insect cells according to a known method. Specially, the method as described in “Current Protocols in Molecular Biology edit. Ausubel et al. (1987) Publish. John Wiley & Sons Section 16.1-16.9” can be followed, for example.

Contacting the substance to be assayed with the cell expressing Reg IIIβ may be carried out by culturing the cells in the presence of the substance to be assayed, or alternatively, it is made possible by directly or indirectly introducing the substance to be assayed into the cells.

The culture medium or culture gas phase in which the cells expressing Reg IIIβ are cultured may be a medium or a gas phase capable of maintaining or culturing the cells. Antibiotics, growth factors and others may be added to those media if necessary.

As used in the present specification, the term “high concentration glucose” may vary depending on the cells expressing Reg IIIβ and their culturing conditions, and it means the glucose concentration at which the reculturing is carried out in the presence of PACAP and which allows an increase in the expression level of Reg IIIβ to be confirmed. If the evaluation method described above is carried out at said glucose concentration, it will be possible to evaluate a substance capable of inhibiting the hyperplasia of pancreatic cells and/or the hypersecretion of insulin by pancreatic cells with high accuracy.

In the culturing of cells expressing Reg IIIβ, when INS-1 cells (rat pancreatic 1 cell line) are employed for example, an RPMI 1640 medium (GIBCO) containing 10 mM HEPES, 10% FBS, 1 mM sodium pyruvate, 50 μM β-mercaptoethanol, 100 units/ml penicillin, 100 μg/ml streptomycin may be used to carry out culturing in an incubator at 5% CO₂/95% air and 37° C. When the culturing is conducted in the presence of glucose at a concentration of 25 mM or more, it is possible to use the increase in the expression level of Reg IIIβ as an index and to evaluate a substance capable of inhibiting the hyperplasia of pancreatic cells and/or the hypersecretion of insulin by pancreatic cells with high accuracy.

This invention also provides a kit for performing the evaluation methods as described above comprising a cell expressing Reg IIIβ, a reagent adapted to contacting a substance to be assayed with the cell, a reagent adapted to culturing the cell in the presence of high concentration glucose and pituitary adenylate cyclase-activating peptide. The use of the kit allows the screening for a substance capable of inhibiting the hyperplasia of pancreatic cells and/or the hypersecretion of insulin by pancreatic cells with rapidness, convenience, and high accuracy.

The substances to be assayed for use in the evaluation method of this invention include, for example, peptides, proteins, non-peptide compounds, synthetic compounds, fermentation products, cell extracts, plant extracts, and animal tissue extracts. These compounds may be either novel compounds or known compounds. Peptide libraries, compound libraries or the like may also be used.

Since the substances screened according to the evaluation method of this invention posses the inhibitory activity for the hyperplasia of pancreatic cells and/or the hypersecretion of insulin by pancreatic cells, they can alleviates the hypofunction of pancreatic cells resulting from the progression of diabetes.

Therefore, if the peptide of this invention is used concomitantly with a known anti-diabetic agent (e.g., a biguanide drug, an α-glucosidase inhibitor, a thiazoline derivative and a sulfonyl drug), it will be possible to prevent or treat diabetes (especially, type 2 diabetes) while inhibiting the hyposecretion of insulin.

Particularly in combination with an agent that has the function of promoting the secretion of insulin such as a SU drug, side effects can be reduced and the combination can be used as a safe, low toxic agent for the prevention or treatment of diabetes.

The compounds to be obtained by the evaluation method may be salts. The salts include salts with physiologically acceptable inorganic acids (e.g., hydrochloric acid and phosphoric acid), organic acids (e.g., acetic acid, formic acid, propionic acid, fumaric acid maleic acid, succinic acid, tartaric acid, citric acid, malic acid, oxalic acid, benzoic acid, methanesulfonic acid and benzenesulfonic acid), or bases (e.g., alkaline metal). Particularly preferred are physiologically acceptable acid-addition salts.

When the compound is used as a pharmaceutical composition, it may be formulated into tablets, capsules, elixir or microcapsules, or alternatively, into sterilized solutions or suspensions for injections according to conventional techniques.

The dosage of the compound will vary depending on the subject to be administered, the method of administration and others. For example, in the case of oral administration to a diabetic patient weighing 60 kg, the dosage will be from about 0.1 to about 100 mg per day, preferably from about 1.0 to about 50 mg, and more preferably from about 1.0 to about 20 mg. In the case of parenteral administration to a diabetic patient weighing 60 kg, intravenous injection may be done at a dosage of from about 0.01 to about 30 mg per day, preferably from about 0.1 to about 20 mg, and more preferably from about 0.1 to about 10 mg.

This invention will be more fully described by way of the examples; however, the invention should not be limited to these examples.

### EXAMPLES

### Example 1

### Construction of Experimental Animals

The construction of experimental animals was carried out according to the description of JP2003304778A. A transgenic mouse having the genetic background of the C57BL/6J mouse (+/+) (Shimizu Laboratory Supplies Co. Ltd.) and overexpressing PACAP specifically in the pancreatic cell (Tg/+; Diabetes (2003) 52:1155-1162) and a KKA^{y} mouse (Clea Japan, Inc.) that was a diabetic model mouse were crossed to produce F₁ mice of the first hybrid generation (+/+, Tg/+, A^{y}/+, Tg/+:A^{y}/+) Four kinds of genotypes were determined by their hair colors (mouse having the A^{y} gene displays yellow body hair color) and PCR utilizing genomic DNAs extracted from their tails. All the animals were bread under the breeding conditions of a temperature of 22° C.±1° C. and an illuminating hour of 12 hours (8:00 to 20:00) and fed with ad lib feeds and water. The breeding conditions and the experiments were all conducted according to the Animal Experiment Guidelines set by Osaka university.

### Experiment 2

### Measurement of Blood Parameters

Blood was collected from the mouse-tail veins between 9:00 to 11:00 am. Serum glucose, insulin and triglyceride levels were measured using a Glucose CII-Test (Wako Pure Chemical Industries, Ltd.), Sensitive Rat Insulin RIA Kit (Linko Research Inc.), and Triglyceride INT Kit (Sigma Corporation), respectively.

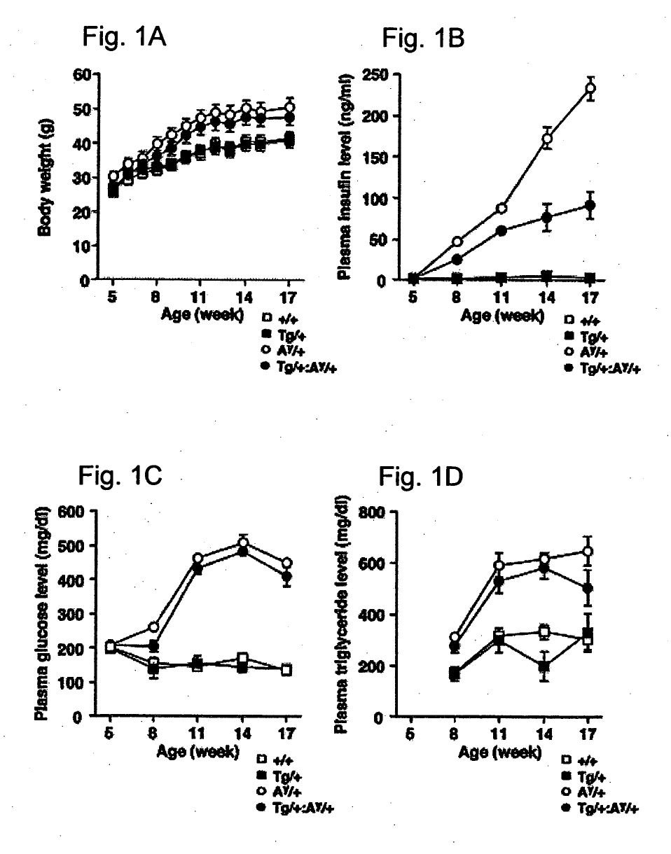

Results are shown in FIG. 1. In the A^{y}/+ mouse and the Tg/+:A^{y}/+ mouse the body weight, the blood glucose level, and the blood triglyceride level drastically increase as the week age and diabetes progressed. However, the increased of the serum insulin level in the Tg/+:A^{y}/+ mouse that had overexpressed PACAP was suppressed to about 40% of the level in the A^{y}/+ mouse. It was thus shown that the introduction of Tg allele partially improved hyperinsulinemia induced by A^{y} allele.

### Example 3

### Quantitative Determination of Tissue Forms

An extracted pancreas was immersed in PBS containing 4% paraformamide for one day and in PBS containing 30% sucrose for two days. After it was frozen in liquid nitrogen, it was preserved at −80° C. until its slices were prepared. A series of slices with a width of 16 μm were prepared and subjected to hematoxylin-eosin (HB) staining.

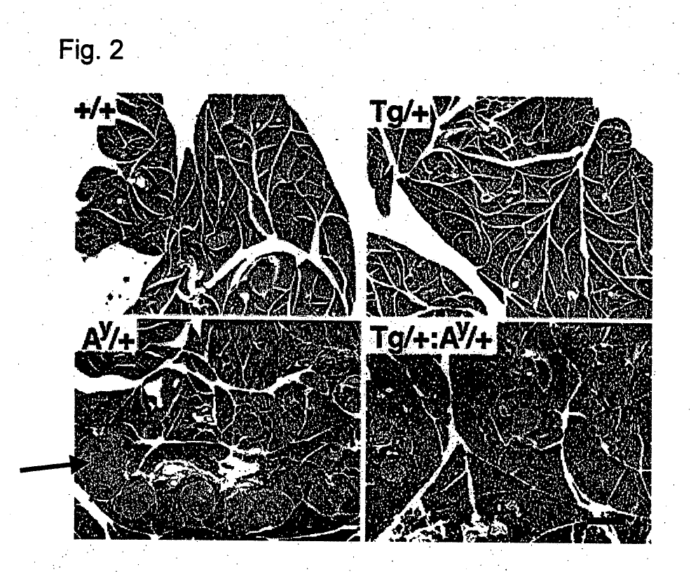

Results are shown FIG. 2. The islets of the A^{y}/+ mouse displayed drastic hyperplasia as compared to those of the wild type. By contrast, the Tg/+:A^{y}/+ mice showed the tendency of contraction in the areas of their islets as compared to the A^{y}/+ mice.

Further, a Nikon TE300 microscope (Nikon Inc.) and a Mac Scope image analysis software (Mitani Co. Ltd.) were used to determine the number and the area of islets on the pancreas slices, and their statistical analysis was carried out.

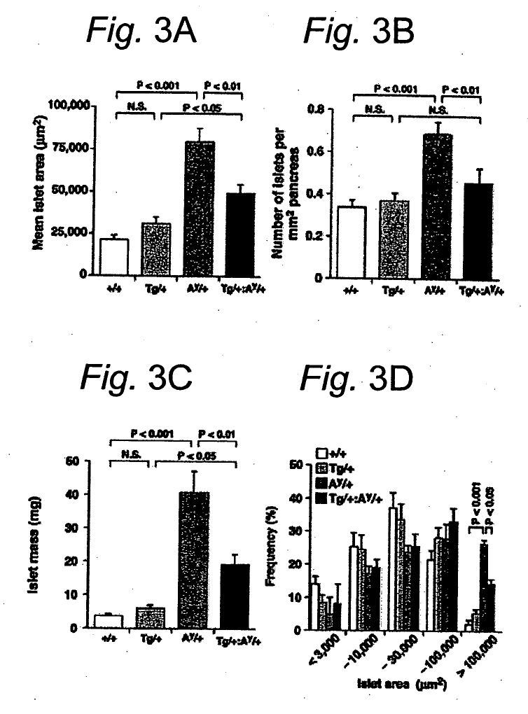

Results are shown in FIG. 3. The mean islet area (FIG. 3A), the number of islets per unit area of the whole pancreas (FIG. 3B), and the islet mass (a value obtained by multiplying the ratio of all islets per the whole pancreas area with the wet weight of the pancreas) (FIG. 3C) all drastically increased in the A^{y}/+ mice as compared to the +/+ mice and the Tg/+ mice. All, however, were suppressed in the Tg/+:A^{y}/+ mice.

The pancreatic insulin contents were lower in the Tg/+:A^{y}/+ mice than in the A^{y}/+ mice by about 61%: they were 13.7±3.7 and 5.4±1.7 μg/g pancreas, respectively. This was consistent with the fact that the increase in the serum insulin level was suppressed in the Tg/+:A^{y}/+ mice.

When histogram analysis was conducted on the sizes of the individual islets (FIG. 3D), the +/+ and Tg/+ mice displayed the histogram patterns of normal distribution while the obese mice having an A^{y}/+ allele displayed distribution patterns shifted to the right, and it was made clear that the mice displayed a drastic increase in the number of islets particularly in an area of greater than 100,000 μm².

The introduction of Tg/+ significantly reduced the number of islets in an area of greater than 100,000 m² that was characteristic of A^{y}/+.

Through a series of histochemical analyses described above, it was found that the overexpression of PACAP in islets inhibited the hyperplasia of the islets in the KKA^{y} mouse of type 2 diabetic condition.

### Example 4

### (1) RNA Extraction from Islets and RNA Amplification

A pancreatic slice was adhered to a non-coat slide glass and was fixed by treatment with 75% EtOH for 30 seconds at −18° C. The glass was immersed in DEPC-treated water for 30 seconds, in 75% EtOH for 30 seconds, in 95% EtOH for 30 seconds, in 100% EtOH for 30 seconds, and in xylene for 7 minutes at room temperature; and it was air-dried for 15 minutes. After it was dried in a desiccator at reduced pressure for 15 minutes, it was preserved in a sample box containing silica gel. A PixCell IIe Laser Capture Microdissection System (Arcturus Bioscience, Inc.) was used to cut out 50 to 70 pieces at the central part of the islet containing β cells abundantly and laser-captured to a CapSure™ (Arcturus Bioscience, Inc.). A Pico Pure RNA Isolation Kit (Arcturus Bioscience, Inc.) was used to prepare total RNA and the first RNA amplification was carried out by using a RiboAmp™ OA RNA Amplification Kit (Arcturus Bioscience, Inc.). The second amplification was carried out on the samples of the first amplified product for which it had been determined by Spot assay that 200 ng or more of the first amplified product was present. CDNA was synthesized from the first amplified product RNA using Superscript II (Invitrogen Corporation) and Oligo-dT T7 primers were used to prepare two types of cDNA. An Enzo High Yield RNA Transcript Labeling Kit (Enzo Diagnostics Inc.) was used to prepare biotinylated cRNA.

### (2) Gene Chip Analysis

Hybridization of the prepared cRNAs to a U74Av2 Gene Chip (Affymetrix, Inc.) was carried out. After the Gene Chip was automatically washed with a Fluidics System (Affymetrix, Inc.) and stained with streptavidin-phycoerythrin, it was scanned with a Hewlett-Packard Gene Array Scanner. Gene transcription level (average difference) was determined by a Microarray Analysis Suite Software (Affymetrix, Inc.) using a Data Image File. In the present analysis one chip was used for one mouse and three mice were used for each genotype. Micro Excel was used to calculate the mean value of gene transcription levels (average differences) in each spot for the genotype.

Four types of F¹ mice were compared among them and the genes with an over 50-fold increase in the average difference value are shown in Table 1. When the +/+ mouse and the A^{y}/+ mouse were compared, the up regulations of carboxypeptidase H, islet amyloid polypeptide, 7B2 protein and others were found in the A^{y}/+ mouse. When the A^{y}/+ mouse and Tg/+:A^{y}/+ mouse were compared, the up regulations of Reg IIIβ, rpL7 and aldolase 2 and others were found in the Tg/+:A^{y}/+ mouse.

TABLE 1
Gene Expression Profiling in Islets

| | Average difference | | | | | |
|---|---|---|---|---|---|---|
| Clone ID | +/+ | Tg/+ | Ay/+ | Tg/+:Ay/+ | Gene | Function |

>50 fold (Ay/+ vs +/+)

| | | | | | | |
|---|---|---|---|---|---|---|
| X61232 | 46 | 107 | 2,871 | 2,385 | carboxypeptidase H | insulin synthesis |
| M25389 | 5 | 37 | 1,213 | 1,042 | Islet amyloid polypeptide | Amyloid formation |
| X61232 | 3 | 14 | 696 | 270 | carboxypeptidase H | Insulin synthesis |
| X15830 | 9 | 25 | 531 | 379 | 7B2 protein | Insulin synthesis |

>50 fold (Tg/+:Ay/+ vs Tg/+)

| | | | | | | |
|---|---|---|---|---|---|---|
| AV371861 | 22 | 18 | 44 | 3,139 | Reg III β | Growth factor |
| M29015 | 21 | 23 | 547 | 1,408 | rpL7 | Protein synthesis |
| D63359 | 9 | 2 | 29 | 1,302 | Reg III β | Growth factor |
| AI527354 | 17 | 14 | 58 | 816 | aldolase 2 | Metabolism |

### Example 5

### Quantitative Analysis of Gene Expression in Mouse Islets

Reverse transcriptase, Superscript II (Invitrogen Corporation) was used to prepare cDNA from the RNA obtained by the second amplification of the islet-derived RNA. Real-time quantitative PCR was conducted with a DNA Engine Opticon 2 System using these cDNA samples as templates, primers specific to each particular gene and a DyNAmo™ SYBR Green qPCR Kit. The reaction conditions employed 40 cycles at 95° C. for 10 seconds, 57° C. for 20 seconds, and 72° C. for 20 seconds. The expression level of the glyceraldehyde 3-phosphate dehydrogenase (GAPDH) gene was analyzed as an internal standard in a similar manner. In PCR the primers having the following sequences were used.

RegIIIβ

forward,

CTG CCT TAG ACC GTG CTT TC (SEQ ID NO:2)

reverse,

CCC TTG TCC ATG ATG CTC TT (SEQ ID NO:3)

Carboxy peptidase H:

forward,

CAC GTA AAT ACA CCC GGA CA (SEQ ID NO:4)

reverse,

TTT CTT CCC ATC CAA AGT GC (SEQ ID NO:5)

IAPP:

forward,

CTC TCT GTG GCA CTG AAC CA (SEQ ID NO:6)

reverse,

GGA CTG GAC CAA GGT TGT TG (SEQ ID NO:7)

GAPDH:

forward,

CTC ATG ACC ACA GTC CAT GC (SEQ ID NO:8)

reverse,

CAC ATT GGG GGT AGG AAC AC (SEQ ID NO:9)

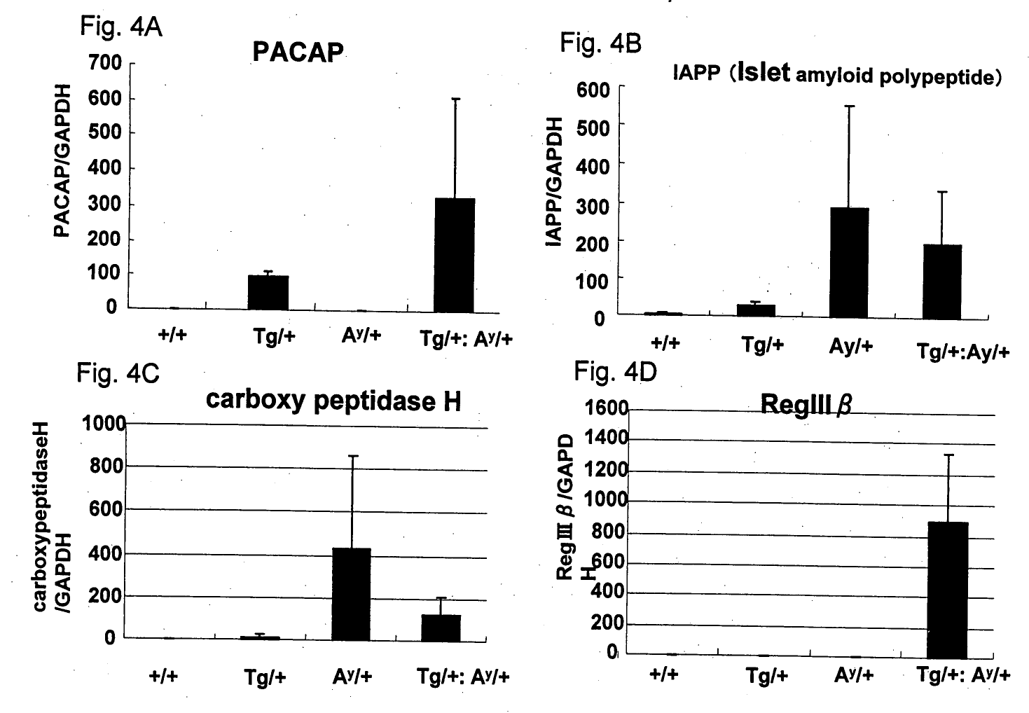

Results are shown in FIG. 4. The same results as in the Gene Chip Analysis were confirmed by the real-time quantitative RT-PCR. When the expression of PACAP was analyzed as a positive control, the mRNA expression enhanced in Tg/+ and Tg/+:A^{y}/+ as compared to in +/+ and A^{y}/+.

The same results as in the Gene Chip analysis were obtained as far as carboxypeptidase H (FIG. 4C), islet amyloid polypeptide (IAPP) (FIG. 4B) and pancreatitis-associated protein (Reg IIIβ) (FIG. 4D) were concerned. This quantitatively confirmed the changes in gene expression obtained in the present experiment.

In the present experiment system there was no gene whose expression level changed in the A^{y}/+ mouse and reached in the Tg/+:A^{y}+ mouse at the same level as in the +/+ mouse. It was, therefore, shown that the Tg allele accomplished the inhibition of the hyperplasia of islets by inducing another gene de novo rather than by normalizing the gene variation caused by the Ay allele.

### Example 6

### (1) INS-1 Cell Culture

INS-1 cells (rat pancreatic β cell line) were cultured in an incubator at 5% CO₂/95% air and 37° C. using an RMI 1640 medium (GIBCO) containing 10 mM HEPES, 10% FBS, 1 mM sodium pyruvate, 50 μM β-mercaptoethanol, 100 units/ml penicillin, and 100 μg/ml streptomycin. Subculturing was carried out every 7 days, using PBS containing 0.25% trypsin/0.02% EDTA

### (2) Analysis of Cell Growth

The cells were seeded on a 48-well plate at a concentration of 1×10⁵ cells/well and were cultured for 48 hours. After washing the cells with PBS, the cells were cultured in an RPMI-1640 medium (test medium) for 12 to 15 hours which had been deprived of glucose and serum and to which 0.1% BSA had been added. Glucose was added to test media containing different concentrations of PACAP so that the glucose was present in the media at 0, 3, 11 and 25 mM, respectively. The cells were cultured in the media for 20 hours, and after further addition of [³H] thymidine (Amersham) to the media in order to achieve a radioactivity of 1 μCi/well, the cells were cultured for additional 4 hours. After washing the cells with PBS twice followed by with 10% trichloroacetic acid (TCA) twice, the cells were fixed by TCA treatment for 30 minutes. The cells were solublized with 0.1 N NaOH and the radioactivity of each medium was measured with a liquid scintillation counter.

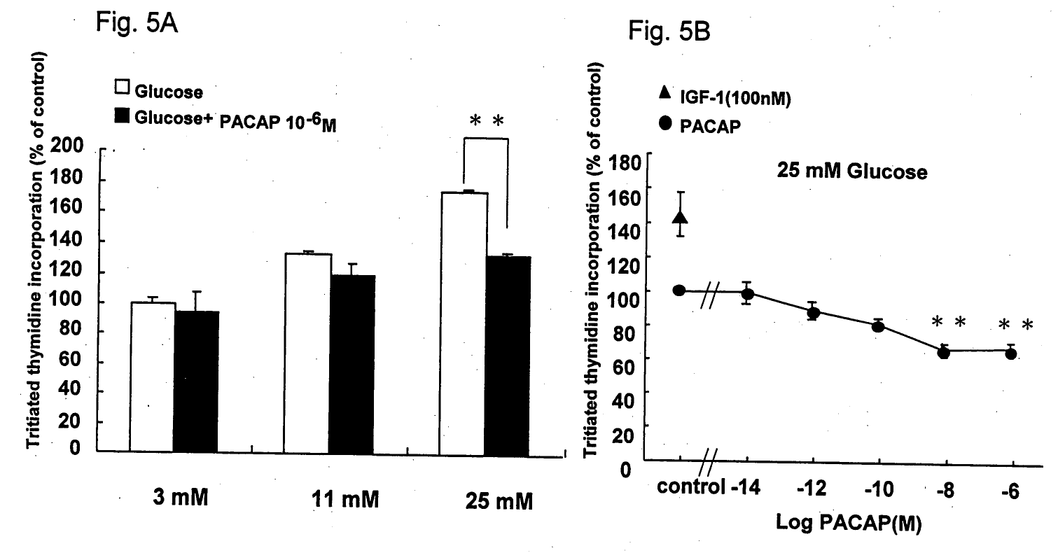

Results are shown in FIG. 5. No effects on cell growth by PACAP were observed under glucose concentration conditions of 3 mM and 11 mM; however, the cell growth inhibition by PACAP at a level of about 20% was observed under the conditions of high glucose concentration (25 mM) (FIG. 5A). It was also shown that PACAP inhibited the cell growth of INS-1 cells in a concentration-dependent manner under the conditions of high glucose concentration (FIG. 5B).

### (3) Quantitative Analysis on Gene Expression of INS-1 Cells

According to the acidic guanidine thiocyanate phenol method, total RNA was prepared from INS-1 cells treated with PACAP; and cDNA was then prepared with reverse transcriptase M-MLV (GIBCO BRL). Real-time quantitative PCR was conducted under the conditions similarly to the mouse islet case. The expression level of the β-actin gene was analyzed as an internal standard in a similar manner. In PCR the primers having the following sequences were used.

RegIIIβ:

forward,

GCT TCA TTC TTG GCA TCC AT (SEQ ID NO:10)

reverse,

AGA TGG GTT CCT CTC CCA GT (SEQ ID NO:11)

β-actin:

forward,

ACC CAC ACT GTG CCC ATC TA (SEQ ID NO:12)

reverse,

GCC ACA GGA TTC CAT ACC CA (SEQ ID NO:13)

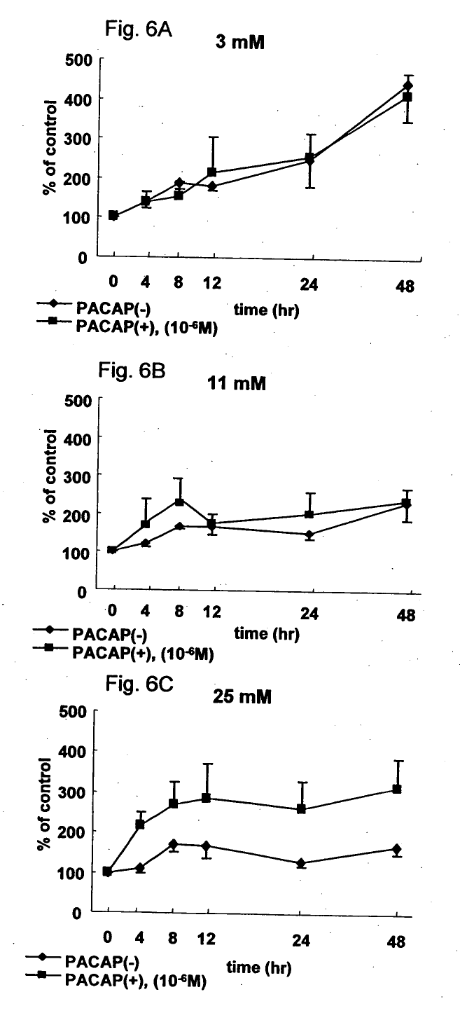

Results are shown in FIG. 6. When expression was quantified by real-time quantitative RT-RCR, no change in expression by the PACAP addition was observed under glucose concentration conditions of 3 mM and 11 mM.

However, the expression-induction of Reg IIIβ by PACAP was observed after 4 hours under the conditions of high glucose concentration (25 mM), and then, a continuous increase in the expression was observed until after 48 hours. Under the glucose conditions of 3 mM, mRNA expression of Reg III β suddenly enhanced regardless of the presence of PACAP after 24 hours. This is thought to be due to that the INS-1 cells were damaged under the low glucose conditions and Reg III β which was a growth factor, was induced.

Based on these results, it is thought that under the conditions of high glucose concentration PACAP specifically upregulates Reg IIIβ and acts on the growth of the pancreatic/cells in a inhibitory manner.

This invention allows the prevention or treatment of a disease associated with the hyperplasia of pancreatic cell and/or the hypersecretion of insulin by pancreatic cells. Further, the invention allows the alleviation of the side effects resulting from an anti-diabetic agent such as a SU drug and is able to provide an excellent anti-diabetic agent.

It will also be possible to evaluate or screen an agent effective for the prevention or treatment of the disease associated with the hyperplasia of pancreatic cells and/or the hypersecretion of insulin by pancreatic cells.

What is claimed is:

1. A method for inhibiting the hyperplasia of pancreatic cells and/or the hypersecretion of insulin by pancreatic cells in a human showing such symptom and in need of inhibition thereof, the method comprises administering to the human, an inhibition effective amount of pituitary adenylate cyclase-activating peptide.

2. An inhibitor of the hyperplasia of pancreatic cells and/or the hypersecretion of insulin by pancreatic cells comprising pituitary adenylate cyclase-activating peptide.

3. A pharmaceutical composition for inhibiting the hyperplasia of pancreatic cells and/or the hypersecretion of insulin by pancreatic cells comprising an effective amount of pituitary adenylate cyclase-activating peptide and a pharmaceutically acceptable carrier.

4. A method for preventing or treating a patient afflicted with diabetes and in need of such treatment or a patient suspected of developing diabetes, the method comprising administering to the patient, pituitary adenylate cyclase-activating peptide and one or more agents selected from a biguanide drug, an α-glucosidase inhibitor, a thiazoline derivative or a sulfonylurea drug.

5. The method according to claim 4, wherein the pituitary adenylate cyclase-activating peptide and the agent are administered simultaneously, separately or consecutively.

6. The method according to claim 4 or 5, wherein the diabetes is type 2 diabetes.

7. A prophylactic or therapeutic agent for diabetes comprising pituitary adenylate cyclase-activating peptide and one or more agents selected from a biguanide drug, an α-glucosidase inhibitor, a thiazoline derivative or a sulfonylurea drug.

8. A pharmaceutical composition comprising pituitary adenylate cyclase-activating peptide characterized by being used simultaneously, separately or consecutively together with a pharmaceutical composition comprising as the effective agent, one or more agents selected from a biguanide drug, an α-glucosidase inhibitor, a thiazoline derivative or a sulfonylurea drug in the prevention or treatment of diabetes.

9. A method for evaluating a substance capable of inhibiting the hyperplasia of pancreatic cells and/or the hypersecretion of insulin by pancreatic cells, the method comprising contacting a substance to be assayed with a cell expressing Reg IIIβ and employing as an index, any change in the expression of Reg IIIβ upon the contact.

10. The method according to claim 9, wherein the method comprises the steps of:
(a) contacting the substance to be assayed with the cell expressing Reg IIIβ;
(b) reculturing the cell contacted with the substance to be assayed in step (a) in the presence of high concentration glucose; and
(c) examining the expression level of Reg IIIβ or the growth rate of the cell expressing Reg IIIβ after reculturing in step (b).

11. The method for evaluation according to claim 10, wherein in step (b) the culturing is carried out in the presence of pituitary adenylate cyclase-activating peptide when the substance to be assayed and the cell expressing Reg IIIβ are recultured.

12. A kit for performing the method for evaluation according to any of claims 9 to 11 comprising a cell expressing Reg IIIβ, a reagent adapted to contacting a substance to be assayed with the cell, a reagent adapted to culturing the cell in the presence of high concentration glucose and pituitary adenylate cyclase-activating peptide.

13. An inhibitor of the hyperplasia of pancreatic cells and/or the hypersecretion of insulin by pancreatic cells comprising a substance obtained by the method for evaluation according to any of claims 9 to 11.

14. A prophylactic or therapeutic agent for diabetes comprising a substance capable of inhibiting the hyperplasia of pancreatic cells and/or the hypersecretion of insulin by pancreatic cells obtained by the method for evaluation according to any of claims 9 to 11 and one or more agents selected from a biguanide drug, an α-glucosidase inhibitor, a thiazoline derivative or a sulfonylurea drug.

15. The inhibitor of the hyperplasia of pancreatic cells and/or the hypersecretion of insulin by pancreatic according to claim 13 characterized by being used simultaneously, separately or consecutively together with a pharmaceutical composition comprising as the effective agent, one or more agents selected from a biguanide drug, an α-glucosidase inhibitor, a thiazoline derivative or a sulfonylurea drug in the prevention or treatment of diabetes.


SEQUENCE LISTING (13 sequences)

SEQ ID NO: 1 - protein, 38 aa, Homo sapiens
  MOD_RES (38)..(38): Amidation
HSDGIFTDSYSRYRKQMAVKKYLAAVLGKRYKQRVKNK

SEQ ID NO: 2 - DNA, 20 bp, Artificial
  Synthetic DNA
ctgccttagaccgtgctttc

SEQ ID NO: 3 - DNA, 20 bp, Artificial
  Synthetic DNA
cccttgtccatgatgctctt

SEQ ID NO: 4 - DNA, 20 bp, Artificial
  Synthetic DNA
cacgtaaatacacccggaca

SEQ ID NO: 5 - DNA, 20 bp, Artificial
  Synthetic DNA
tttcttcccatccaaagtgc

SEQ ID NO: 6 - DNA, 20 bp, Artificial
  Synthetic DNA
ctctctgtggcactgaacca

SEQ ID NO: 7 - DNA, 20 bp, Artificial
  Synthetic DNA
ggactggaccaaggttgttg

SEQ ID NO: 8 - DNA, 20 bp, Artificial
  Synthetic DNA
ctcatgaccacagtccatgc

SEQ ID NO: 9 - DNA, 20 bp, Artificial
  Synthetic DNA
cacattgggggtaggaacac

SEQ ID NO: 10 - DNA, 20 bp, Artificial
  Synthetic DNA
gcttcattcttggcatccat

SEQ ID NO: 11 - DNA, 20 bp, Artificial
  Synthetic DNA
agatgggttcctctcccagt

SEQ ID NO: 12 - DNA, 20 bp, Artificial
  Synthetic DNA
acccacactgtgcccatcta

SEQ ID NO: 13 - DNA, 20 bp, Artificial
  Synthetic DNA
gccacaggattccataccca